SEQUENCE LISTING (10 sequences)

SEQ ID NO: 1 - DNA, 23 bp, Artificial
  Sense primer
gtacatcccccactggaaaaatt

SEQ ID NO: 2 - DNA, 23 bp, Artificial
  Sense primer
gtacatcccccactggaaaaata

SEQ ID NO: 3 - DNA, 17 bp, Artificial
  Antisense primer
caccacgcccggctaag

SEQ ID NO: 4 - DNA, 18 bp, Artificial
  Antisense primer
gcaccacgcccggctaat

SEQ ID NO: 5 - DNA, 19 bp, Artificial
  Sense primer
gctcagctgaacctggcta

SEQ ID NO: 6 - DNA, 18 bp, Artificial
  Sense primer
ctcagctgaacctggctg

SEQ ID NO: 7 - DNA, 18 bp, Artificial
  Reverse primer
acagagccagccaagcca

SEQ ID NO: 8 - DNA, 22 bp, Artificial
  Sense primer
ccacttagttatccagatcctc

SEQ ID NO: 9 - DNA, 22 bp, Artificial
  Sense primer
ccacttagttatccagatcctt

SEQ ID NO: 10 - DNA, 25 bp, Artificial
  Reverse primer
gctttgatccagatatgtattacac

(19) United States
(12) Patent Application Publication
(10) Pub. No.: US 2004/0170996 A1
(43) Pub. Date: Sep. 2, 2004

(54) Cytotoxic T-Lymphocyte antigen-4 or interleukin-10 polymorphisms as predictors of response to therapeutic intervention

(76) Inventors: Leland Yee, Pittsburgh, PA (US); Jianming Tang, Birmingham, AL (US); Richard A. Kaslow, Birmingham, AL (US); Dirk J. van Leeuwen, Birmingham, AL (US)

Correspondence Address: GIFFORD, KRASS, GROH, SPRINKLE, ANDERSON & CITKOWSKI, PC, 280 N OLD WOODARD AVE, SUITE 400, BIRMINGHAM, MI, 48009, US

(21) Appl. No.: 10/469,277

(22) PCT Filed: Feb. 27, 2002
(86) PCT No.: PCT/US02/06207

Related U.S. Application Data
(60) Provisional application No. 60/271,811, filed on Feb. 27, 2001.

(51) Int. Cl. C12Q 1/70; C12Q 1/68

(52) U.S. Cl. 435/6; 435/5

(57) ABSTRACT

A composition and process are provided for predicting the responsiveness of an individual to therapy for a pathological condition is based on detecting nucleic acid allele sequences in an interleukin-10 regulatory region or cytotoxic t-lymphocyte antigen-4 promoter or exon region of the individual. The detected nucleic acid allele is then associated with the level of therapeutic responsiveness to a pathological condition based upon genotype. Comparison across multiple alleles and regions improves the predictive nature of the process.

### FIELD OF INVENTION

The present invention relates generally to a composition and process for identify and analyzing genetic polymorphisms in an interleukin-10 regulatory region and/or cytotoxic T-lymphocyte antigen-4 that have utility in predicting an individual's response to therapeutic intervention with interferon-α-2b and ribavirin for hepatitis C virus (HCV) infection.

### BACKGROUND

Interleukin-10 is a potent anti-inflammatory T-helper type 2 cytokine that downregulates the expression of MHC class I and class II molecules as well as the production of T-helper type 1 cytokines. (Fiorentino et al. J. Immunol. 1991; 146:3444-3451; Yue et al. Int. J. Cancer 1997; 71:630-637; Tsuruma et al. Cell Immunol. 1998; 184:121-128; Kundu et al. Cell Immunol. 1997; 180:55-61; Knolle et al. Clin. Exp. Immunol. 1998; 114:427-433; de Waal et al. J. Exp. Med. 1991; 174:915-924; de Waal et al. J. Exp. Med. 1991; 174:1209-1220.) Varying levels of molecules that function in inflammatory and immune responses are thought to play a prominent role in the heterogeneous course of many diseases and infections. (Yee et al. Genes and Immunity 2000; 1:386-390; Tang et al. Genes and Immunity 1999; 1:20-27). It is known that interleukin-10 levels differ among individuals. (Eskdale et al. Proc. Natl. Acad. Sci. USA 1998; 95:9465-9470; Eskdale et al. Genes and Immunity 1999; 1:151-155.) Interleukin-10 production plays a role in the pathogenesis of a number of infectious as well as autoimmune diseases, which include but are not limited to: hepatitis C infection, human immunodeficiency virus (HIV) infection and rheumatoid arthritis, systemic lupus erythematosus, and graft-versus-host disease in allogeneic bone marrow transplantation (Bidwell et al. 1999 Genes and Immunity; 1:3-19).

HCV infection is an example of a condition whose pathogenesis and course varies with the individual infected, and interleukin-10 levels may play a role in this heterogeneity. Treatment of hepatitis C infection currently relies heavily on the combination of interferon-α and ribavirin. Virologic sustained response to interferon-α and ribavirin, defined as undetectable HCV RNA at 6 months after discontinuation of therapy, is achievable in only 30-60% of treated patients. (Poynard et al. Lancet 1998; 352:1426-1432; McHutchison et al. N. Engl. J. Med. 1998; 339:1485-1492.) For many of those who undergo this therapy, a variety of often debilitating side effects may be encountered, which may include severe hemolytic anemia and debilitating fatigue. (Dusheiko. Hepatology 1997; 26:112S121S; Okanoue et al. J. Hepatol. 1996; 25:283-291.) These therapeutic disadvantages make it desirable to predict response or non-response to treatment in order to avoid unnecessary morbidity, as well as the expense of unsuccessful treatment. There has been no reliable method for predicting sustained response of an individual to interferon-α-2b and ribavirin treatment of hepatitis C virus infection. The shortcomings of this regimen have prompted a search for factors that could predict those most likely to benefit from it. Candidate predictors now include viral genotypes, age, race, and early disappearance of HCV-RNA after initiation of therapy. (Brouwer et al. J. Hepatol. 1999; 30:192-198.) Racial differences in response rates to IFN+R have been highlighted, with the vast majority of African-Americans being non-responders (NRs) to interferon-based therapy. (McHutchison et al. Gastroenterology 2000; 119:1317-1323; Reddy et al. Hepatology 1999; 30:787-793.) It has been shown that the interleukin-10 regulatory region haplotype ATA, corresponding to the −592, −819 and −1082 polymorphic positions, respectively on the IL-10 gene, is associated with initial responsiveness to interferon therapy and that the GCC haplotype (also corresponding to the same polymorphic sites) is associated with non-responsiveness to interferon therapy. (Edwards-Smith et al. Hepatology 1999; 30:526-530.) Initial responsiveness is defined as loss of hepatitis virus C RNA and normalization of serum alanine transaminase levels after 12 weeks of interferon treatment (Edwards-Smith et al. Hepatology 1999; 30:526-530.) However, it is known that a proportion of initial responders relapse after cessation of interferon therapy (Lee et al. Hepatology 1998; 28:1411-1415). Thus there is a current need for a means of predicting sustained response to treatment for hepatitis C virus infection.

As a potent immune modulator, interleukin-10 may exert a profound impact on the overall therapeutic outcome. High serum levels of interleukin-10 have been correlated with poor response to interferon therapy, while interleukin-10 production has been found to be lower in responders than in non-responders. (Kuzushita et al. Scand. J Gastroenterol. 1997; 32:169-174; Cramp et al. Gastroenterology 2000; 118:346-355.) Ribavirin itself has also been shown to downregulate interleukin-10 production in several in vitro studies (Hultgren et. al. J. Gen. Virol. 1998; 79:2381-2391; Tam et al. J. Hepatol. 1999; 39:376-382) and may contribute to the greater efficacy of interferon and ribavirin in achieving a sustained virologic HCV response compared with interferon monotherapy. (Cramp et al. Gastroenterology 2000; 118:346-355.) Furthermore, development of chronic HCV infection has been associated with a high level of interleukin-10 and a TH2-type cytokine profile. (Tsai et al. Hepatology 1997; 25:449-458.) Overall, it is clear that elevated levels of interleukin-10 are not favorable for HCV patients.

Polymorphisms in the interleukin-10 gene contribute to the differences in interleukin-10 levels observed between individuals. (Eskdale et al. Proc. Natl. Acad. Sci. US4 1998; 95:9465-9470; Eskdale et al. Genes and Immunity 1999; 1:151-155.) Specifically, three single nucleotide polymorphisms in the interleukin-10 regulatory region at positions −1082, −819 and −592 relative to the transcription start site, form three single nucleotide polymorphism combinations, ATA, ACC, and GCC, that have been associated with differential interleukin-10 expression. (Eskdale et al. Proc. Natl. Acad. Sci. USA 1998; 95:9465-9470; Eskdale et al. Genes and Immunity 1999; 1:151-155; Edwards-Smith et al. Hepatology 1999; 30:526-530.)

The cytotoxic T-lymphocyte antigens (CTLA-4), encoded by a gene on chromosome 2q33, is expressed on activated CD4+ and CD8+T-cells. (Dariavach et al. Eur. J. Immunol. 1988; 18:1901-5.) It binds to the ligands B7-1 and B7-2 (Linsley et al. Immunity 1994; 1:793-801; Walunas et al. Immunity 1994; 1:405-13; Lindsten et al. J. Immunity 1993; 151:3489-99) and is believed to down-regulate T-cell function (Walunas et al. Immunity 1994; 1:405-13; Kristiansen et al. Genes Immun. 2000; 1:170-184; Chambers et al. Proc. Natl. Acad. Sci. USA 1999; 96:8603-8; Krummel et al. J. Exp. Med., 1995; 182:459-65; Sperling et al. Immunol. Rev. 1996; 153:155-82). Mice deficient in CTLA-4 exhibit polyclonal T-cell activation and proliferation. (Waterhouse et al. Science 1995; 270:985-8; Chambers et al. Proc. Natl. Acad. Sci. USA 1997; 94:9296-301; Tivol et al. Immunity 1995; 3:541-7.) A polymorphism resulting in a C-to-T transition at position −318 of the CTLA-4 promoter has also been described. (Deichmann et al. Biochem. Biophys. Res. Commun. 1996; 225:817-8.). In addition, a G-to-A transition at position 49 in exon-1 of the CTLA-4 gene encodes an alanine (Ala)-to-threonine (Thr) substitution in codon 17 of the leader peptide. (Larsen et al. Autoimmunity 1999; 31:35-42; Nistico et al. Hum. Mol. Genet. 1996; 5:1075-80.) Thus, there is a need in the treatment of chronic HCV infection to determine whether these single nucleotide polymorphisms (SNP) and CTLA-4 are associated with response to interferon-α-2b and ribavirin.

### BRIEF DESCRIPTION OF THE DRAWINGS

FIG. 1 shows polymorphic sites in the interleukin-10 regulatory region (approximately to scale) located on chromosome 1 and compares allele and haplotype frequencies.

### SUMMARY OF THE INVENTION

A process for predicting a therapeutic response in an individual includes detecting a first nucleic acid allele in an interleukin-10 regulatory region, cytotoxic T-lymphocyte antigen promoter or exon region of the individual. The first allele is then compared with a second allele from the corresponding region that is associated with a known outcome upon interferon and ribavirin administration to a given pathological condition. A correlation between alleles in an interleukin-10 regulatory region and in a cytotoxic T-lymphocyte antigen promoter or exon optionally increases the predictive process with respect to a therapeutic response.

A process for predicting a therapeutic response in an individual includes detecting a first nucleic acid allele in an interleukin-10 regulatory region or cytotoxic T-lymphocyte antigen promoter or exon region of the individual and comparing the first allele with a second nucleic acid allele of the corresponding region, the second allele associated with sustained response to therapeutic intervention in a pathological condition. The use of oligonucleotide primers for detection of an interleukin-10 regulatory region or cytotoxic T-lymphocyte antigen-4 promoter or exon region to predict sustained response to therapeutic intervention for a pathological condition is provided. Additionally, the use of these oligonucleotide primers to predict an individual's responsiveness to interferon and ribavirin treatment of a pathological condition is also provided. A commercial kit for prediction of response in an individual to interferon and ribavirin treatment of a pathological condition includes reagents for detecting a first nucleic acid allele in an interleukin-10 regulatory region or a cytotoxic T-lymphocyte antigen-4 promoter or exon region of the individual and comparing the first allele with the second allele in the corresponding region, where the second allele is associated with a known outcome of therapeutic administration. The kit further includes instructions for the use of the reagents therefor.

### DETAILED DESCRIPTION OF THE PREFERRED EMBODIMENTS

The present invention is a process for predicting the responsiveness of an individual to therapy for a pathological condition by detecting a nucleic acid allele sequence in an interleukin-10 regulatory region or cytotoxic T-lymphocyte antigens (CTLA-4) promoter or exon region of an individual. The detected nucleic acid allele is then associated with responsiveness or non-responsiveness to therapy for a pathological condition In particular, the present invention provides a process to evaluate responsiveness of an individual infected with hepatitis C virus to interferon-α-2b and ribavirin treatment, including determining the nucleic acid allele in the interleukin-10 regulatory region of the individual and comparing the detected nucleic acid allele with those shown by this invention to be associated with sustained response or non-response to interferon-α-2b and ribavirin treatment.

Prior to the present invention, the only known association between interleukin-10 regulatory region polymorphisms and responsiveness to therapy for hepatitis C virus infection concerned individual responsiveness to the initial 12 weeks of such therapy. (Edwards-Smith et al. Hepatology 1999; 30:526-530.) While this is an important association, more important aspects of responsiveness to therapy for hepatitis C virus infection are apparent only after the therapy is discontinued. When therapy is discontinued, a proportion of individuals experience virologic relapse where levels of HCV return, as before therapy. (Lee et al., Hepatology 1998; 28:1411-1415.) The present invention shows that interleukin-10 or polymorphisms are associated with sustained response to therapy and therefore allows selection of appropriate individuals for therapy by reliable prediction of sustained response.

The present invention is also distinguished over previous contributions to this field in that it predicts a sustained response to the most effective current therapy for hepatitis C virus infection, a combination interferon-α-2b and ribavirin. Previous work has shown an association between interleukin-10 polymorphisms and initial response interferon-α treatment for hepatitis C virus infection. (Edwards-Smith et al. Hepatology 1999; 30:526-530.) However, it is known that interferon-α in combination with ribavirin is more effective than treatment with ribavirin alone (28:1411-1415; Poynard et al. Lancet 1998; 352:1426-1432; McHutchison et al. N. Engl. J. Med. 1998; 339:1485-1492). Furthermore, the present invention relies on the identification of a specific subset of the ATA haplotype being involved with sustained response, namely the haplotype between the IL.10.R 108 base-pair allele and the ATA proximal haplotype. Therefore, the prediction of responsiveness to interferon-α alone is of limited value since the standard of care is with interferon +ribavirin combination therapy.

The present invention is based on the novel association of interleukin-10 regulatory region polymorphisms, alone or in combination with polymorphisms in CTLA-4, with likelihood of success in treatment of hepatitis C infection with interferon-2b and ribavirin. Thus the present invention has utility as a process for identifying infected individuals who will respond to interferon-α-2b and ribavirin treatment for hepatitis C virus infection, thus saving non-responders the discomfort and expense of such treatment.

The term “allele” or “allelic form” is intended to mean an alternative version of a gene with the same function but containing differences in nucleotide sequence relative to another version of the same gene.

The term “allelic polymorphism” or “allelic variant” is intended to mean a variation in the nucleotide sequence within a gene, wherein different individuals in a general population express different variants of the gene.

The term “allelic pattern” is intended to mean the identity of each of two copies of a particular gene in a patient, i.e. homozygosity or heterozygosity.

The term “genotype” is used herein interchangeably with “allelic pattern”.

The term “haplotype” is intended to mean a combination of alleles that are found in a single chromosome and tend to be inherited together.

The term “genotyping” as used herein as being the process of determining the allelic patterns of an individual human.

The term “oligonucleotide primer” is intended to mean a molecule comprised of two or more deoxyribonucleotides, preferably more than three although the exact size will depend on many factors, that is capable of acting as a point of initiation of DNA synthesis when placed under appropriate conditions. The oligonucleotide may be derived synthetically or by cloning.

While it is clear that the preferred embodiment of the present invention has utility in predicting response to treatment in hepatitis C virus infection, it is acknowledged that because interleukin-10 and CTLA-4 are important modulators of the immune response each individually, the present invention also has utility in predicting the outcome of therapeutic intervention in pathological conditions in mammals illustratively including, rheumatoid arthritis, systemic lupus erythematosus, Sjogren's syndrome, inflammatory bowel disease, Felty's syndrome, allergies, asthma, myasthenia gravis, systemic vasculitis, glomerulonephritides, cancer, multiple sclerosis, Chagas disease, meningococcal infection and Toxoplasma gondii infection.

The present invention identifies the association between interleukin-10 regulatory region polymorphisms and sustained response to interferon-α-2b and ribavirin therapy for hepatitis C infection. A separate correlation is identified for CTLA-4 and exon 1 thereof with the same therapy. Presence of the −592A or −819T single nucleotide polymorphisms; the −592 A/A or −819 T/T genotypes; the combination of −592A/−819T as a haplotype; homozygosity for −592A/−819T//−592A/−819T as a genotype; possession of the (108)TCATA haplotype. The addition of the IL10.R. suggests that it is a specific lineage (subset) of the proximal ATA haplotype that is responsible for therapeutic response. While having a single copy of this haplotype is associated with sustained response, all individuals homozygous for this haplotype, achieved sustained response. In contrast, the (108)TCACC haplotype is associated with non-response to this therapy. The present invention provides a method for detecting the nucleotide allelic form at positions −592, −819, −1082, −2763, and −3575 in the interleukin-10 regulatory region by testing DNA from an individual.

Optionally, additional testing is performed on the −318 CTLA-4 promoter and 49 exon 1 allelic positions. The detection of the individual's allelic form is achieved using polymerase chain reaction (PCR)—based amplification. (Koss et al. Genes and Immunity 2000; 1:321-324.) It is appreciated that other techniques known in the art may also be employed, these illustratively include the use of allele-specific oligonucleotides as hybridization probes and/or as primers for DNA amplification, cloning and sequencing of amplified DNA and restriction enzyme analysis of DNA.

Further, the DNA used in the predictive test provided by this invention may be obtained from any cell source or body fluid containing intact nucleic acid. Most simply, DNA can be extracted from cells of the blood in a sample drawn from an individual.

The present invention also encompasses analysis of the detected allele of the interleukin-10 regulatory region and optionally the CTLA-4 and exon 1 thereof for the individual patient in order to predict the likelihood of success in treating HCV infection with interferon-α-2b and ribavirin. The analysis involves comparison of the detected allele with alleles associated with sustained response or non-response to interferon-α-2b and ribavirin treatment of HCV infection, as follows: presence of the −592A or −819T single nucleotide polymorphisms; the −592 A/A or −819 T/T genotypes; the combination of −592A/−819T as a haplotype; homozygosity for −592A/−819T//−592A/−819T as a genotype; possession of the (108)TCATA haplotype and homozygosity for this haplotype is associated with a higher likelihood of sustained response, while the (108)TCACC haplotype is associated with non-response.

The present invention provides compositions of oligonucleotides to be used in detection of polymorphisms in the interleukin-10 regulatory regions or CTLA-4 promoter or exon. These oligonucleotides anneal with specific regions of the interleukin-10 regulatory region in the DNA extracted from the patient sample, this annealing being an essential step in polymerase chain reaction amplification of DNA.

The present invention also provides a kit for detection of a polynucleotide comprising a portion of the interleukin-10 or CTLA-4 gene in a human sample, said kit comprising oligonucleotide primers complimentary to a part of the interleukin-10 or CTLA-4 regulatory region and instructions for their use. As a first step in the use of such a kit, DNA is extracted from an individual's cells by a process well known to those skilled in the art. The individual's DNA is mixed with reagents for amplification of DNA by polymerase chain reaction. These reagents typically include an appropriate buffer, a mix of deoxyribonucleotides, MgC12, two oligonucleotide primers complimentary to the part of the interleukin-10 regulatory region to be amplified and an appropriate amount of a thermostable polymerase. This mixture is incubated at temperatures and for times well known to those skilled in the art. The amplified DNA can then be analyzed by a number of techniques, including but not limited to, gel electrophoresis or cloning and sequencing. The kit is useful for clinicians and patients so that they can decide whether to proceed with interferon-α-2b and ribavirin therapy. This decision is based on the predictions of therapeutic success derived from comparison of the detected nucleic acid allelic form with nucleic acid allelic forms associated with sustained response or non-response to interferon-α-2b and ribavirin treatment.

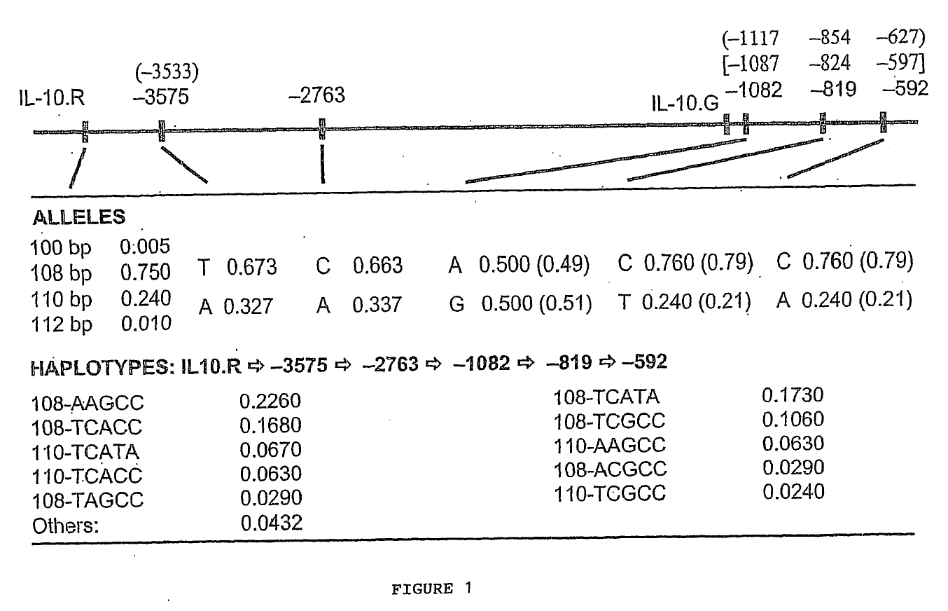

Definition of interleukin-10 regulatory region polymorphisms predicts response to interferon-α-2b and ribavirin therapy. Presence of the −592A or −819T single nucleotide polymorphisms; the −592 A/A or −819 T/T genotypes; the combination of −592A/−819T as a haplotype; homozygosity for −592A/−819T//−592A/−819T as a genotype; possession of the (108)TCATA haplotype and homozygosity for this haplotype is associated with a higher likelihood of sustained response. In contrast, the (108)TCACC haplotype is associated with non-response. This is shown in analysis of data obtained by genotyping HCV patients of whom 49 are sustained responders and 55 are non-responders. No significant differences are detected between sustained responders and non-responders with respect to the distribution of their age, gender, pre-treatment alanine aminotransferase values or pre-treatment viral load levels. Frequencies of proximal regulatory region single nucleotide polymorphisms and microsatellite variants ranged from 0.005 to 0.760, as shown in FIG. 1. Genotype distributions at all polymorphic sites approximate Hardy-Weinberg equilibrium. Examination of homozygous genotypes combined with contingency tests leads to the identification of several haplotypes as shown in FIG. 1.

Differences in several interleukin-10 allele, genotype and haplotype distributions between sustained responders and non-responders are observed. The −592A and −819T alleles, along with the exclusively linked −592 A/A and −819 T/T genotypes, are more frequent in sustained responders than in non-responders as shown in Table 1. These two sites are dimorphic and reciprocal effects (non-response) are also seen with the −592C and −819C alleles. Homozygosity for genotypes −592 A/A and −819T/T is more strongly associated with sustained response than heterozygosity as shown in Table 1.

TABLE 1

| | | | |
|---|---|---|---|
| SR (%) | NR (%) | OR | p-value |

Allele

| | | | | |
|---|---|---|---|---|
| −819 C | 68.4 | 82.7 | 0.45 | 0.02 |
| −819 T | 31.6 | 17.3 | 2.2 | 0.02 |
| −592 C | 68.4 | 82.7 | 0.45 | 0.02 |
| −592 A | 31.6 | 17.3 | 2.2 | 0.02 |

Genotype

| | | | | |
|---|---|---|---|---|
| −819 C/C | 49 | 65.5 | 0.51 | 0.09 |
| −819 C/T | 38.8 | 34.6 | 1.2 | 0.66 |
| −819 T/T | 12.2 | 0 | 16.6 | 0.01 |
| −592 A/A | 12.2 | 0 | 16.6 | 0.01 |
| −592 C/A | 38.8 | 34.5 | 1.2 | 0.66 |
| −592 C/C | 49 | 65.5 | 0.51 | 0.09 |

Haplotype (IL10.R [symbol] −3575 [symbol] −2763 [symbol] −1082 [symbol] −819 [symbol] −592)

| | | | | |
|---|---|---|---|---|
| (108)TCATA | 24.5 | 10.9 | 2.65 | 0.01 |
| (108)TCACC | 10.2 | 22.7 | 0.39 | 0.02 |

Heterozygosity for haplotype

| | | | | |
|---|---|---|---|---|
| (108)TCATA | 28.6 | 21.8 | 1.4 | 0.43 |

Homozygosity for haplotype

| | | | | |
|---|---|---|---|---|
| (108)TCATA | 10.2 | 0 | 13.7 | 0.02 |

As shown in Table 1, the (108)TCATA extended haplotype is also associated with sustained response (OR=2.7; p=0.01) and homozygosity for this haplotype (N=5 individuals) shows an exclusive relationship with sustained response (OR=13.7; p=0.0253). Carriage of the (108)TCATA haplotype does not affect how quickly responders clear HCV RNA (by week 4 or 12). The other major haplotype involving the ATA proximal single nucleotide polymorphisms, combination (110)TCATA, is observed and is equally distributed among the sustained responders (7.1%) and sustained responders groups (6.4%) (OR=1.1; p=0.8233). In contrast, the (108)TCACC haplotype is associated with non-response.

As shown in FIG. 1, the interleukin-10.R and interleukin-10.G microsatellite loci and 3 single nucleotide polymorphisms in the proximal regulatory region regions are identified in at least 3 different numbering systems (Koss et al. Genes and Immunity 2000; 1:185-190; Eskdale et al. Genes and Immunity 2000; 1:151-155; and Turner et al. Eur. J. Immunogenetics 1997; 24:1-8). Also in FIG. 1, a comparison of allele frequencies is given (Turner et al., Eur. J. Immunogenet. 24:1-8). Two additional polymorphic sites are shown, including a C/A exchange at −2763 and a T/A exchange at −3575. The −3575 polymorphism has also been numbered −3533 by D'Alfonso, et al. (D'Alfonso et al. Genes and Immunity 2000; 1:231-233.) Together, the microsatellite and single nucleotide polymorphism variants can form several stable haplotypes, which are also provided in FIG. 1.

CTLA-4 polymorphisms complement the current array of predictors of therapeutic response to interferon-α-2b and ribavirin, including interleukin-10 polymorphisms. Genotype-1 infection in Caucasians with sustained response showed a consistent association of the 49G allelic variant and the GIG genotype. The relationships with this genetic specificity appeared independent of both potential confounders (such as age and gender) and other established cofactors for response such as baseline viral load and IL-10 polymorphism.

Treated carriers of the 49G variant in CTLA-4 exon-1 reached lower levels of viremia more rapidly than non-49G carriers, which indicates the biologic/clinical significance of the inventive screening process. Similar beneficial effects were not seen with the −318 promoter polymorphism by itself, but tight linkage between the −318C promoter and the 49G variants accounted for an equally strong effect of the −318C+49G haplotype in virologic sustained responders.

The apparent advantage of the 49G allele or the two-locus haplotype −318C+496 was confined to infection with genotype-1 virus in Caucasians. Genotype-1 viruses differ intrinsically from the others in the magnitude or quality of the T-cell response they elicit. Missale and colleagues examined whether viral genotype can exert an influence on the modulatory effect of interferon (IFN) on antiviral T-cells. (Missale et al. Hepatology 1997; 26:792-797.) Their observations suggest a different interferon α-2b effect on T-cell responses among individuals infected with genotype-1 virus compared to those with non-I virus; those infected with non-I virus had stronger interferon a-2b-induced T-cell responses to HCV core antigen. (Missale et al. Hepatology 1997; 26:792-797.)

Due to the extreme distributions of the individuals homozygous for the interleukin-1 (108) TCATA haplotype (N=5 SR and N=0 NR), the interleukin-10 (108) TCATA phenotype was used as a surrogate marker and observed an even stronger effect for the interleukin-10 (108) TCATA phenotype (OR=2.7; p=0.0979) when controlling for the effects of CTLA-4 than that shown in Table 1. Associations of both interleukin-10 (108) TCATA phenotype and the CTLA-4 allele/haplotype retain their separate effects in multivariable analysis.

The present invention is further illustrated by the following non-limiting scope examples.

### EXAMPLE 1

### Study Subjects

HCV patients enroll in a trial of interferon-α-2b and ribavirin to be included in this study. The selection criteria for the trial have been previously described. (Polynaid et al. Lancet 1998; 352:1426; McHutchison et al. N. Engl. J. Med. 1998; 339:1485.) Briefly, all participants have: 1) compensated liver disease due to HCV infection, 2) no other form of chronic liver disease; and 3) no infection with either the human immunodeficiency virus UV) or hepatitis B virus (HBV). For each patient, HCV genotypes are assessed along with serum HCV RNA levels at the time of enrollment, at weeks 4 and 12 after treatment, and at 6 months after discontinuation of therapy. Sustained response is defined as an undetectable level of HCV RNA at 6 months after discontinuation of therapy. Non-responders have a level of >25,000 copies/mL of HCV RNA at week 12 or have an initial week 12 response but a detectable viral level 6 months after discontinuation of therapy.

### EXAMPLE 2

### DNA Extraction and PCR-Based Genotyping of Interleukin-10 Variants

Genomic DNA is extracted from whole blood of participants using standard inorganic salt-out procedures (Miller, et al.; Nucleic Acids Res 1988; 16:1215 and Grimberg et al.; Nucleic Acids Res 1989; 17:8390). Polymerase chain reaction with sequence specific primers (PCR-SSP) is used to define the interleuldn-10 regulatory region single nucleotide polymorphisms at the −592, −819 and −1082 positions as reported. (Koss et al. Genes and Immunity 2000; 1:321-324.) These sites are also known as −1117, −854, and −627, respectively. (Eskdale et al. Proc. Natl. Sci. USA 1998; 95:9465-9470.) Two other single nucleotide polymorphisms in the distal regulatory region at positions −2763 and −3575, relative to the transcription start site are similarly typed using the following primers: IL10-3575TF (sense primer specific for −3575T, 5′-gTA CAT CCC CCA CTg gAA AAA TT-3′, SEQ ID NO:1), IL10-3575AF (sense primer specific for −3575A, 5′-gTA CAT CCC CCA CTg gAA AAA TA-3′, SEQ ID NO:2), IL10-2763CR (antisense primer specific for −2763C, 5′-CAC CAC gCC Cgg CTA Ag-3′, SEQ ID NO:3), and IL10-2763AR (antisense primer specific for −2763A, 5′-gCA CCA CgC CCg gCT AAT-3′, SEQ ID NO:4). The −3575 site corresponds to the recently reported −3533 site. (D'Alfonso et al. Genes and Immunity 2000; 1:231-233.) Interleukin-10.R microsatellite alleles are amplified by PCR and separated on denaturing gels with the 5′ primer labeled with Cys to allow automated detection on the ALFexpress DNA sequencer (Amersham Pharmacia Biotech, Piscataway, N.J.). (Eskdale et al. Immunogenetics 1997; 46:120-128.)

### EXAMPLE 3

### DNA Extraction and PCR-Based Genotyping of CTLA-4 Variants

Genomic DNA is extracted from whole blood using standard inorganic salt-out procedures of Example 2. Polymerase chain reaction with sequence specific primers (PCR-SSP) was used to define the SNP in position 49 of exon-1 in the CTLA-4 gene. Primers consisted of CTLA-4-SSP-1 (specific for the position 48A allele): 5′-GCTCAGCTGAACCTGGCTA-3′ (SEQ ID NO:5) and —CTLA-4-SSP-2: (specific for the position 49G allele): 5′-CTCAGCTGAACCTG GCTG-3′ (SEQ E) NO:6) and a common reverse primer, CTLA-4-SSP-G: 5′-ACAGAGCCAGCCAA GCCA-3′ (SEQ ID NO:7). Primers for the −318 SNP consisted of SSP-3:5′-CCA CTTAgTTATCCAgA TCCTC-3′ (SEQ ID NO:8) (forward and −318C-specific), SSP-4:5′-CCACTTAgT-TATCCAgATCCTT-3′ (SEQ ID NO.9) (forward and −318T-specific), and 5′-gCTTTg ATCCAg ATATgTATTACAC-3′ (SEQ ID NO:10) (reverse and general primer). Control primers specific for DRB1 or Tapasin are used in all reactions to generate amplicons to assess the reliability of each assay. PCR solution (10 μl each) consisted of 1× buffer C (60 mM Tris-HCI, pH 8.5, 15 mM (NH₄)₂SO₄, 2.5 MM MgCl₂), 50-70 ng of geniomic DNA, 0.3 units of AmpliTaq polymerase, 120 nM of each control primer, 250 nM each of TAP1-specific primer, 0.4 mM each of dGTP, dCTP, dTTP and dATP, 10% (v/v) glycerol, and 0.02% cresol red. PCR cycling began with 10 higher-stringency cycles of denaturing at 95° C. for 25 sec, annealing at 62° C. for 45 sec, and extension at 72° C. for 45 sec, followed by 22 additional lower-stringency cycles of denaturing at 95° C. for 25 sec, annealing at 58° C. for 40 sec, and extension at 72° C. for 40 sec. Half of each PCR reaction product is loaded directly onto 1.5% agarose gels for electrophoresis, and the SSP-banding patterns are recorded on photographs of ethidium bromide-stained gels.

### EXAMPLE 4

### Statistical Analyses

All statistical analyses, including X² tests, odds ratios, and p-values (both maximum likelihood and Fisher's Exact) are conducted using the SAS® software (Cary, N.C., USA).

### EXAMPLE 5

### Distribution of CTLA-4 promoter and Exon 1 Allelic Variants

The frequencies of the CTLA-4 promoter and exon 1 genotypes in Caucasians approximated Hardy-Weinberg equilibrium (for the promoter: C/C: 83.6%; C/T: 14.6%; T/T: 1.8%; for exon-1: G/G: 10.4%; G/A: 44.8%; A/A: 44.8%). There were too few African-Americans studied herein to expect a close fit to the Hardy-Weinberg model: (for the promoter, genotype distributions were: C/C: 100%; C/T: 0%; T/T: 0%; and for exon-1: G/G 33.3%; G/A 33.3%; A/A 53.4%).

Among Caucasians with genotype 1 infections, 39 were sustained responders and 73 were non-sustained responders, while among Caucasians with genotype non-1 infections, 40 were sustained responders and 6 were non-sustained responders. Caucasian sustained responders with genotype-1 infections carried only a slightly higher proportion of the promoter −318C variant in univariate analysis, as shown in Table 2.

TABLE 2
Differential distribution of CTLA-4 allele, phenotype and genotype
frequencies for the −318 SNP, 49 SNP, along with the −318 + 49
haplotype, among Caucasian sustained responders (SR) and
non-responders (NR) infected with genotype-1 viruses.

| | | | | | |
|---|---|---|---|---|---|
| MARKER | SR | NR | OR | 95% C.I. | P-VALUE |

Allele Frequency [2N = 76 SR; 2N = 146 NR]*

| | | | | | |
|---|---|---|---|---|---|
| −318C | 72 | 128 | 2.5 | 0.82-7.8 | 0.0953 |
| −318T | 4 | 18 | 0.40 | 0.13-1.2 | 0.0953 |
| 49G | 30 | 34 | 2.1 | 1.1-3.7 | 0.0170 |
| 49A | 48 | 112 | 0.49 | 0.3-0.9 | 0.0170 |
| −318C + 49G | 29 | 33 | 2.1 | 1.2-3.8 | 0.0145 |
| −318C + 49A | 43 | 95 | 0.70 | 0.40-1.2 | 0.2170 |
| −318T + 49A | 4 | 17 | 0.42 | 0.14-1.3 | 0.1240 |

Allele Carrier (Phenotype) Frequency [N = 39 SR; N = 73 NR]*

| | | | | | |
|---|---|---|---|---|---|
| −318T | 4 | 15 | 2.3 | 0.70-7.4 | 0.1687 |
| 49G | 25 | 32 | 2.3 | 1.0-5.1 | 0.0420 |
| 49A | 34 | 71 | 0.19 | 0.04-0.90 | 0.0370 |
| −318C + 49G | 24 | 31 | 2.3 | 1.0-5.2 | 0.0394 |
| −318T + 49A | 4 | 15 | 0.46 | 0.14-1.5 | 0.1855 |

Genotype Frequency [N = 38 SR; N = 73 NR]*

| | | | | | |
|---|---|---|---|---|---|
| −318C/C | 34 | 58 | 2.2 | 0.69-7.1 | 0.1850 |
| 49G/G | 5 | 2 | 5.2 | 1.0-28.3 | 0.0366 |
| 49A/A | 14 | 41 | 0.44 | 0.2-1.0 | 0.0419 |
| −318C + 49G/ | 5 | 2 | 5.4 | 1.0-29.2 | 0.0451 |
| −318C + 49G | | | | | |
| −318C + 49A/ | 12 | 33 | 0.56 | 0.25-1.3 | 0.1672 |
| −318C + 49A | | | | | |

NOTE:
Only markers with a p < 0.25 are listed.
*For the −318 SNP: N = 38 SR (2N = 76) and N = 73 NR (2N = 146).

For the −318 SNP: N=38 SR (2N=76) and N=73 NR (2N=146). For the position 49 SNP in exon-1, the 49G variant was significantly more frequent in sustained responders (OR=2.1; p=0.017); frequencies of allele carriers (phenotype) and homozygous genotypes were also significantly more frequent among sustained responders (OR=2.3; p=0.042) and (OR=5.2; p=0.037), respectively). The dimorphic nature of the −318 and 49 SNPs produced reciprocal effects (inhibition of response) for the −318T and 49A variants and carrier frequencies. The 49G effects were not observed in Caucasian patients infected with non-1 genotype viruses (p>0.25 for all).

### EXAMPLE 6

### CTLA-4 Haplotype Analyses

Strong linkage disequilibrium is observed between the promoter and exon-1 variants, as shown in Table 3 and previously reported. (Donner et al. Diabetes 1998;47:1158-1160.)

TABLE 3
Observed linkage disequilibrium between
Promoter and Exon-1 polymorphisms.

| −318 | | | | | | |
|---|---|---|---|---|---|---|
| SNP | 49 SNP | HFreq | Delta value | OR | p-value | 95% C.I. |
| T | A | 7.4 | 0.02746 | 22.4 | <0.0001 | 3.0-165.9 |
| T | G | 2.9 | 0.00618 | 1.5 | 0.2975 | 0.70-3.1 |
| C | G | 28.9 | 0.05013 | 21.0 | <0.0001 | 5.1-87.0 |
| C | A | 53.6 | 0.03967 | 2.9 | <0.0001 | 1.8-4.9 |

Abbreviations:
HFreq = haplotype frequency;
OR = Odd's Ratio;
95% C.I. = 95% Confidence Interval.

Frequencies of the −318C+49G haplotype counted individually and by carrier were significantly higher in sustained responders as shown in Table 2. The association of homozygosity for the haplotype was even stronger in sustained responders. Corresponding associations were not seen among Caucasian patients infected with non-1 genotype viruses (p>0.25 for all).

### EXAMPLE 7

### Multivariable Analyses Between Interleukin-10 and CTLA-4

Control for effect of homozygosity for the interleukin-10 (108)TCATA haplotype is precluded by the small number of individuals with this haplotype (N=5). As a surrogate, possession of the IL-10 (108)TCATA phenotype was accounted for. The associations of the 49G variant in Caucasian SRs with genotype-1infections persisted after adjustment in multivariable analysis as shown in Table 4 for gender, age at treatment, interaction between age at treatment and gender, baseline viral load, baseline ALT and possession of the interleukin-10 (108)TCATA phenotype. (Hayashi et al. Arch. Intern. Med. 1998; 158:177-181.)

TABLE 4
Multivariate analyses of the effects of the CTLA-4-(G) phenotype
among Caucasian patients with genotype-1 infections.

| MULTIVARIATE MODEL | OR | 95% C.I. | P-VALUE |
|---|---|---|---|
| 49-(G) phenotype | 2.6 | 1.1-6.0 | 0.0320 |
| baseline viral load | 0.78 | 0.33-1.8 | 0.5731 |
| baseline ALT | 1.4 | 0.59-3.5 | 0.4198 |
| gender | 2.4 | 0.81-7.4 | 0.1128 |
| age at treatment | 0.34 | 0.02-5.7 | 0.4559 |
| IL-10 (108)-TCATA | 2.7 | 0.83-8.7 | 0.0979 |
| phenotype | | | |
| interaction between gender | 1.2 | 0.20-6.8 | 0.8754 |
| and age | | | |

Interaction between carriage of 49G and of interleukin-10 (108)TCATA is not detected (data not shown).

### EXAMPLE 8

### Longitudinal Analyses for CTLA-4

Among the Caucasian SRs with genotype-1 infections, an assessment is made as to whether carriage of the 49G allele resulted in an effect on viral dynamics during the initial 12 weeks of therapy, as shown in Table 5. Significant differences in viral load are detected between 49G carriers and non-carriers. Viral RNA concentration appeared to decline more rapidly in carriers; lower levels were seen in carriers at each successive point.

TABLE 5
Longitudinal analysis. Mean viral load measurements (log₁₀)
at baseline, week 4 and 12 of therapy are presented along
with the F-statistic value (repeated measures) and p-value 1)
for differences in the change of viral load during the first 12
weeks of therapy associated with 49G phenotype (Column 1),
and 2) for interaction between phenotype with time (Column 2)
among Caucasian patients with genotype-1 infections.

| | | | | | | Column 2 | |
|---|---|---|---|---|---|---|---|
| Viral Loads | | | | Column 1 | | 49G Phenotype + | |
| | Week | Week | Week | 49G Phenotype | | Time | |
| Marker | 0 | 4 | 12 | F-statistic | p-value | F-statistic | p-value |
| 49G | 14.4 | 10.1 | 8.1 | 6.62 | 0.0114 | 3.78 | 0.0249 |
| 49A | 14.5 | 11.7 | 9.7 | — | — | — | — |

Any patents or publications mentioned in this specification are indicative of the levels of those skilled in the art to which the invention pertains. These patents and publications are herein incorporated by reference to the same extent as if each individual publication was specifically and individually indicated to be completely incorporated by reference.

One skilled in the art will readily appreciate that the present invention is well-adapted to carry out the objects and obtain the ends and advantages mentioned, as well as those inherent therein. The present examples along with the methods, procedures, treatments, molecules and specific compounds described herein are presently representative of preferred embodiments, are exemplary, and are not intended as limitations on the scope of the invention. It will be apparent that other embodiments exist and are encompassed within the spirit of the invention as defined by the scope of the claims.

What is claimed is:

1. A process for predicting a therapeutic response in an individual comprising the steps of: detecting a first nucleic acid allele in an interleukin-10 regulatory region of the individual; and comparing said first nucleic acid allele with a second nucleic acid allele in said interleukin-10 regulatory region associated with a known outcome of administration of interferon-α-2b and ribavirin in a pathological condition.

2. The process of claim 1 wherein said first and said second alleles have the same sequence.

3. The process of claim 1 wherein said pathological condition is a viral infection.

4. The process of claim 1 wherein said viral infection is hepatitis C virus.

5. The process of claim 4 wherein said hepatitis C virus is selected from a group consisting of hepatitis C variants 1a, 1b and 3a.

6. The process of claim 1 wherein said interleukin-10 gene is a mammalian interleukin-10 regulatory region.

7. The process of claim 1 wherein said interleukin-10 regulatory region is a human interleukin-10 regulatory region.

8. The process of claim 1 wherein said first nucleic acid allele comprises two or more interleukin-10 regulatory region nucleic acids at positions selected from the group consisting of: −592, −819, −1082, −2763 and −3575.

9. The process of claim 1 wherein said second nucleic acid allele is selected from the group consisting of: −592A or −819T single nucleotide polymorphisms; the −592 A/A or −819 T/T genotypes; the combination of −592A/−819T as a haplotype; homozygosity for −592A/−819T//−592A/−819T as a genotype; the (108)TCATA haplotype and the(108)TCACC haplotype.

10. The process of claim 1, further comprising the steps of: detecting a cytotoxic T-lymphocyte antigen-4 promoter or exon allele.

11. The process of claim 1 wherein said cytotoxic T-lymphocyte antigen-4 promoter or exon allele is at a position selected from a group consisting of: −318 and 49.

12. The process of claim 1, wherein detecting said first nucleic acid allele is by amplification of DNA with two oligonucleotide primers selected from the group consisting of: SEQ D NOS: 1-4.

13. The process of claim 1, further comprising the step of collecting DNA from blood obtained from the individual.

14. A process for predicting a therapeutic response in an individual comprising the steps of: detecting a first nucleic acid allele in an interleukin-10 regulatory region of the individual and comparing said first nucleic acid allele with a second nucleic acid allele in said interleukin-10 regulatory region associated with sustained response to therapeutic intervention in a pathological condition.

15. The process of claim 14 wherein said pathological condition is a viral infection.

16. The process of claim 14 wherein said viral infection is hepatitis C virus.

17. The process of claim 16 wherein said hepatitis C virus is selected from a group consisting of hepatitis C variants 1a, 1b and 3a.

18. The process of claim 14 wherein said pathological condition is a bacterial infection.

19. The process of claim 18 wherein said bacterial infection is meningococcus infection.

20. The process of claim 14 wherein said pathological condition is selected from the group consisting of: rheumatoid arthritis, systemic lupus erythematosus, human immunodeficiency virus, hepatitis C and graft versus host disease.

21. The process of claim 14 wherein said therapeutic intervention comprises administration of at least one agent selected from the group consisting of: interferon-α, interferon-β, interferon-γ, interferon-β-1a, interferon-β-1b, interferon-α-2b and ribavirin.

22. The process of claim 12 wherein said interleukin-10 gene is a mammalian interleukin-10 regulatory region.

23. The process of claim 14 wherein said interleukin-10 regulatory region is a human interleukin-10 regulatory region.

24. The process of claim 14 wherein said first nucleic acid allele comprises two or more interleukin-10 regulatory region nucleic acids at positions selected from the group consisting of: −592, −819, −1082, −2763 and −3575.

25. The process of claim 14 wherein said second nucleic acid allele is selected from the group consisting of: −592A or −819T single nucleotide polymorphisms; the −592 A/A or −819 T/T genotypes; the combination of −592A/−819T as a haplotype; homozygosity for −592A/−819T//−592A/−819T as a genotype; the (108)TCATA haplotype and the (108)TCACC haplotype.

26. The process of claim 14, further comprising the steps of: detecting a cytotoxic T-lymphocyte antigen-4 promoter or exon allele.

27. The process of claim 26 wherein said cytotoxic T-lymphocyte antigen-4 promoter or exon allele is at a position is selected from a group consisting of: −318 and 49.

28. The process of claim 14, wherein detecting said first nucleic acid allele is by amplification of DNA with two oligonucleotide primers chosen from the group comprising: SEQ ID NOS:1-4.

29. The process of claim 14, wherein said process further comprises the step of collecting DNA from blood obtained from the individual.

30. A process for predicting a therapeutic response in an individual comprising the steps of: detecting a first allele in a cytotoxic T-lymphocyte antigen-4 promoter or exon region of the individual; and comparing said first allele with a second allele in said cytotoxic T-lymphocyte antigen-4 promoter or exon associated with a known outcome of administration of interferon-α-2b and ribavirin in a pathological condition.

31. The process of claim 30 wherein said first and said second alleles have the same sequence.

32. The process of claim 30 wherein said pathological condition is a viral infection.

33. The process of claim 30 wherein said viral infection is hepatitis C virus.

34. The process of claim 33 wherein said hepatitis C virus is selected from a group consisting of hepatitis C variants 1a, 1b and 3a.

35. The process of claim 30 wherein said cytotoxic T-lymphocyte antigen-4 promoter or exon region is a mammalian cytotoxic T-lymphocyte antigen-4 promoter or exon region.

36. The process of claim 30 wherein said cytotoxic T-lymphocyte antigen-4 promoter or exon region is a human cytotoxic T-lymphocyte antigen-4 promoter or exon region.

37. The process of claim 30 wherein said first allele is at a position selected from the group consisting of: −318 and 49.

38. The process of claim 30 further comprising the steps of detecting a first nucleic acid allele in an interleukin-10 regulatory region of the individual; and comparing said first nucleic acid allele with a second nucleic acid allele in said interleukin-10 regulatory region associated with a known outcome of administration of interferon-α-2b and ribavirin in a pathological condition.

39. The process of claim 38 wherein said first nucleic acid allele comprises two or more interleukin-10 regulatory region nucleic acids at positions selected from the group consisting of: −592, −819, −1082, −2763 and −3575.

40. The process of claim 38 wherein said second nucleic acid allele in the interleukin-10 regulatory region is selected from the group consisting of: −592A or −819T single nucleotide polymorphisms; the −592 A/A or −819 T/T genotypes; the combination of −592A/−819T as a haplotype; homozygosity for −592A/−819T//−592A/−819T as a genotype; the (108)TCATA haplotype and the(108TCACC haplotype.

41. The process of claim 30, wherein detecting said first allele is by amplification of DNA with a primer selected from the group consisting: SEQ ID NOS:5-10.

42. The process of claim 30, further comprising the step of collecting DNA from blood obtained from the individual.

43. A process for predicting a therapeutic response in an individual comprising the steps of: detecting a first allele in a cytotoxic T-lymphocyte antigen-4 promoter or exon region of the individual and comparing said first allele with a second allele in said cytotoxic T-lymphocyte antigen-4 promoter or exon region associated with sustained response to therapeutic intervention in a pathological condition.

44. The process of claim 43 wherein said pathological condition is a viral infection.

45. The process of claim 43 wherein said viral infection is hepatitis C virus.

46. The process of claim 45 wherein said hepatitis C virus is selected from a group consisting of hepatitis C variants 1a, 1b and 3a.

47. The process of claim 43 wherein said pathological condition is a bacterial infection.

48. The process of claim 47 wherein said bacterial infection is meningococcus infection.

49. The process of claim 43 wherein said pathological condition is selected from the group consisting of: rheumatoid arthritis, systemic lupus erythematosus, human immunodeficiency virus, hepatitis C and graft versus host disease.

50. The process of claim 43 wherein said therapeutic intervention comprises administration of at least one agent selected from the group consisting of: interferon-α, interferon-β, interferon-γ, interferon-β-1a, interferon-β-1b, interferon-α-2b and ribavirin.

51. The process of claim 43 wherein said cytotoxic T-lymphocyte antigen-4 promoter or exon region is a mammalian cytotoxic T-lymphocyte antigen-4 promoter or exon region.

52. The process of claim 43 wherein said cytotoxic T-lymphocyte antigen-4 promoter or exon region is a human cytotoxic T-lymphocyte antigen-4 promoter or exon region.

53. The process of claim 43 wherein said first allele is at a position selected from the group consisting of: −318 and 49.

54. The process of claim 43, wherein detecting said first allele is by amplification with two oligonucleotide primers chosen from the group consisting of SEQ ID NOS:5-10.

55. A composition comprising an oligonucleotide primer selected from the group consisting of SEQ ID NOS:1-10.

56. The use of oligonucleotide primers for detection of an interleukin-10 regulatory region or cytotoxic T-lymphocyte antigen-4 promoter or exon allele to predict sustained response to therapeutic intervention for a pathological condition.

57. The use of claim 56 wherein said pathological condition is hepatitis C virus infection.

58. The use of oligonucleotide primers for detection of an interleukin-10 regulatory region or cytotoxic T-lymphocyte antigen-4 promoter or exon allele to predict response to the interferon-α-2b and ribavirin treatment of a pathological condition.

59. The use of claim 58 wherein said pathological condition is hepatitis C virus infection.

60. A commercial kit for prediction of response in an individual to interferon-α-2b and ribavirin treatment of a pathological condition, comprising reagents for the process of claim 1 or 30; and instructions for their use.

61. A commercial kit for prediction of sustained response in an individual to therapeutic intervention in a pathological condition, comprising reagents for the process of claim 12 or 43; and instructions for their use.
